(19) United States
(12) Patent Application Publication
(10) Pub. No.: US 2016/0083345 A1
(43) Pub. Date: Mar. 24, 2016

(54) POLYMORPHIC FORMS OF LOMITAPIDE AND ITS SALTS AND PROCESSES FOR THEIR PREPARATION

(71) Applicant: CADILA HEALTHCARE LIMITED, Ahmedabad (IN)

(72) Inventors: Sanjay Jagdish DESAI, Ahmedabad (IN); Brij KHERA, Ahmedabad (IN); Jagdish Maganlal PATEL, Ahmedabad (IN); Harshita Bharatkumar SHAH, Ahmedabad (IN); Arunkumar Shyam Narayan UPADHYAY, Ahmedabad (IN); Sureshkumar Narbheram AGRAVAT, Ahmedabad (IN)

(73) Assignee: CADILA HEALTHCARE LIMITED, Ahmedabad (IN)

(21) Appl. No.: 14/857,925

(22) Filed: Sep. 18, 2015

(30) Foreign Application Priority Data
Sep. 19, 2014 (IN) 3006/MUM/2014
Feb. 19, 2015 (IN) 541/MUM/2015
Apr. 11, 2015 (IN) 1521/MUM/2015
Jul. 13, 2015 (IN) 2642/MUM/2015

(51) Int. Cl. C07D 211/58; C07C 303/44; C07C 303/32; C07C 309/04

(52) U.S. Cl. CPC C07D 211/58; C07C 309/04; C07C 303/44; C07C 303/32; C07B 2200/13

(57) ABSTRACT

The present invention relates to various polymorphic forms of lomitapide or its salts and processes for preparation thereof. The present invention provides Lomitapide mesylate in solid amorphous form and process for preparation thereof. The invention also provides an amorphous solid dispersion of lomitapide mesylate. Further, various crystalline forms of lomitapide mesylate like A, B and C and process for preparation thereof are provided. The invention also provides crystalline forms of lomitapide free base, in particular Form I and Form-II and their preparation. The invention further provides compositions comprising various forms of lomitapide and its salts.

### FIELD OF THE INVENTION

The field of the invention relates to polymorphic forms of lomitapide or its salts and processes for preparation thereof. In particular, the invention relates to the polymorphic forms of lomitapide free base and lomitapide mesylate. More particular, the present invention relates to an amorphous form and crystalline forms of lomitapide free base and lomitapide mesylate, respectively and processes for their preparation.

### BACKGROUND OF THE INVENTION

The following discussion of the prior art is intended to present the invention in an appropriate technical context and allow its significance to be properly appreciated. Unless clearly indicated to the contrary, however, reference to any prior art in this specification should be construed as an admission that such art is widely known or forms part of common general knowledge in the field.

Lomitapide is a synthetic lipid-lowering agent for oral administration. It is a microsomal triglyceride transfer protein inhibitor approved as Juxtapid® in US and as Lojuxta® in Europe as an adjunct to a low-fat diet and other lipid-lowering treatments, including LDL apheresis where available, to reduce low-density lipoprotein cholesterol (LDL-C), total cholesterol (TC), apolipoprotein B (apo B), and non-highdensity lipoprotein cholesterol (non-HDL-C) in patients with homozygous familial hypercholesterolemia (HoFH). The approved drug product is a mesylate salt of lomitapide, chemically known as N-(2,2,2-trifluoroethyl)-9-[4-[4-[[[4′(trifluoromethyl)[1,1′-biphenyl]-2-yl]carbonyl]amino]-1-piperidinyl]butyl]-9H-fluorene-9carboxamide methanesulfonate [“lomitapide mesylate” herein after] and has the structural formula

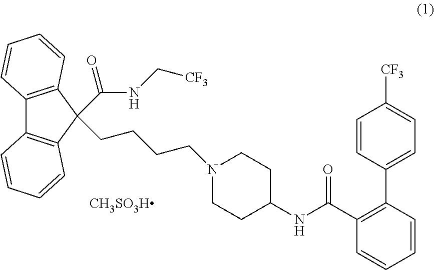 [SMILES: O=C(NC1CCN(CCCCC2(C(=O)NCC(F)(F)F)c3ccccc3-c3ccccc32)CC1)c1ccccc1-c1ccc(C(F)(F)F)cc1]

As per the approved label for Juxtapid® (US) “Lomitapide mesylate is a white to off-white powder that is slightly soluble in aqueous solutions of pH 2 to 5. Lomitapide mesylate is freely soluble in acetone, ethanol, and methanol; soluble in 2-butanol, methylene chloride, and acetonitrile; sparingly soluble in 1-octanol and 2-propanol; slightly soluble in ethyl acetate; and insoluble in heptane”.

As per Public Assessment Report for Lojuxta® (Europe) “Polymorphism has been observed for lomitapide mesylate. Of the different solid-state forms, hydrates, and solvates identified in the polymorph studies, only 2 desolvated solid-state forms, Form I and Form II, were identified in batches after drying to final drug substance.” The report further states, under the heading Manufacture, that “The final particle size distribution is controlled during the crystallisation step” (emphasis added) suggesting that the approved drug product lomitapide mesylate is a crystalline compound.

U.S. Pat. No. 5,712,279 A discloses the lomitapide compound and a process for its preparation. It also discloses a process for preparation of lomitapide monohydrochloride.

U.S. Pat. No. 5,883,109 A discloses lomitapide mesylate specifically but no solid form was disclosed.

The reference article Synthesis and Applications of Isotopically Labelled Compounds, Vol. 8, Pg. 227-230 (2004) discloses the preparation of Deuterium labelled [d₄]BMS-201038, [³H]BMS-201038, [¹⁴C]BMS-201038 wherein BMS-201038 is lomitapide mesylate.

International (PCT) Publication No. WO 2015/121877 A2 discloses lomitapide crystalline Form I and Form II as well as amorphous form of Lomitapide mesylate and processes for their preparation.

There is still a need to provide a novel polymorph of lomitapide or its salts which is suitable for pharmaceutical preparations. Therefore, the present invention provides new crystalline forms of lomitapide free base and lomitapide mesylate. The present invention also provides amorphous form of lomitapide free base and lomitapide mesylate, which is stable and useful for pharmaceutical preparations.

### SUMMARY OF THE INVENTION

In one general aspect, there is provided lomitapide mesylate in solid amorphous form.

In another general aspect, there is provided a crystalline Form-A of lomitapide mesylate.

In another general aspect, there is provided a crystalline Form-B of lomitapide mesylate.

In another general aspect, there is provided a crystalline Form-C of lomitapide mesylate.

In another general aspect, there is provided lomitapide free base in solid amorphous form.

In another general aspect, there is provided a crystalline Form-I of lomitapide free base.

In another general aspect, there is provided a crystalline Form-II of lomitapide free base.

In another general aspect, there is provided a process for the preparation of a lomitapide mesylate, the process comprising reacting lomitapide free base with methane sulfonic acid.

In another general aspect, there is provided a process for the preparation of an amorphous form of lomitapide mesylate, the process comprising:
(a) providing a solution of lomitapide mesylate in one or more solvents; and
(b) obtaining the amorphous form of lomitapide mesylate by the removal of the solvents.

In another general aspect, there is provided a process for the preparation of an amorphous form of lomitapide mesylate, the process comprising:
(a) providing a solution of lomitapide free base in one or more solvents;
(b) adding methane sulfonic acid to the solution; and
(c) obtaining the amorphous form of lomitapide mesylate by the removal of the solvents.

In another general aspect, there is provided a process for the preparation of an amorphous form of lomitapide mesylate, the process comprising:
(a) providing a solution of lomitapide free base in one or more solvents;
(b) adding methane sulfonic acid to the solution; and
(c) obtaining the amorphous form of lomitapide mesylate by adding one or more anti-solvents to the solution.

In another general aspect, there is provided a process for the preparation of a crystalline form of lomitapide mesylate, the process comprising:
(a) providing a solution of lomitapide mesylate in one or more solvents; and
(b) obtaining the crystalline form of lomitapide mesylate by the removal of the solvents.

In another general aspect, there is provided a process for the preparation of amorphous form of lomitapide free base, the process comprising:
(a) providing a solution of lomitapide base in one or more solvents; and
(b) obtaining the amorphous form of lomitapide free base by the removal of the solvents.

In another general aspect, there is provided a process for the preparation of an amorphous form of lomitapide free base, the process comprising:
(a) providing a solution of lomitapide free base in one or more solvents;
(b) cooling the solution; and
(c) obtaining the amorphous form of lomitapide free base by adding one or more anti-solvents to the solution.

In another general aspect, there is provided a process for the preparation of crystalline form of lomitapide free base, the process comprising:
(a) providing a solution of lomitapide free base in one or more solvents; and
(b) obtaining the crystalline form of lomitapide free base by the removal of the solvent.

In another general aspect, there is provided a process for the preparation of crystalline form of lomitapide free base, the process comprising:
(a) providing a solution of lomitapide free base in one or more solvents;
(b) heating the solution; and
(c) obtaining the crystalline form of lomitapide free base by adding one or more anti-solvents to the solution.

In another general aspect, there is provided a pharmaceutical composition comprising an amorphous form of lomitapide mesylate and pharmaceutically acceptable carriers, diluents and excipients.

In another general aspect, there is provided a pharmaceutical composition comprising a crystalline Form-A of lomitapide mesylate and pharmaceutically acceptable carriers, diluents and excipients.

In another general aspect, there is provided a pharmaceutical composition comprising a crystalline Form-B of lomitapide mesylate and pharmaceutically acceptable carrier, diluents and excipients.

In another general aspect, there is provided a pharmaceutical composition comprising a crystalline Form-C of lomitapide mesylate and pharmaceutically acceptable carriers, diluents and excipients.

In another general aspect, there is provided a pharmaceutical composition comprising a crystalline form of lomitapide free base and pharmaceutically acceptable carriers, diluents and excipients.

In another general aspect, there is provided a pharmaceutical composition comprising an amorphous form of lomitapide free base and pharmaceutically acceptable carriers, diluents and excipients.

In another general aspect, there is provided an amorphous form of lomitapide mesylate having a purity of about 98.0% or more, when measured by an area percentage by HPLC.

In another general aspect, there is provided a crystalline Form-A of lomitapide mesylate having a purity of about 98.0% or more, when measured by an area percentage by HPLC.

In another general aspect, there is provided a crystalline Form-B of lomitapide mesylate having a purity of about 98.0% or more, when measured by an area percentage by HPLC.

In another general aspect, there is provided a crystalline Form-C of lomitapide mesylate having a purity of 98.0% or more, when measured by an area percentage by HPLC.

### BRIEF DESCRIPTION OF THE DRAWINGS

FIG. 1: Discloses x-ray powder diffractogram of amorphous form of lomitapide mesylate.

FIG. 2: Discloses x-ray powder diffractogram of a crystalline Form-A of lomitapide mesylate.

FIG. 3: Discloses x-ray powder diffractogram of a crystalline Form-B of lomitapide mesylate.

FIG. 4: Discloses x-ray powder diffractogram of an amorphous form of lomitapide free base.

FIG. 5: Discloses x-ray powder diffractogram of a crystalline Form-I of lomitapide free base.

FIG. 6: Discloses x-ray powder diffractogram of a crystalline Form-II of lomitapide free base.

FIG. 7: Discloses x-ray powder diffractogram of crystalline Form-C of lomitapide mesylate.

FIG. 9: Discloses Differential Scanning Calorimetry of crystalline Form-C of lomitapide mesylate.

FIG. 9: Discloses FT-IR spectrum of crystalline Form-C of lomitapide mesylate.

### DETAILED DESCRIPTION OF THE INVENTION

The above objectives of the present invention are achieved by the disclosures and description provided herein after.

The ranges recited herein, if any, for any experimental parameter are not absolute and a reasonable degree of expected experimental, instrumental, technical and/or human error in such measurements should be considered.

As used herein the term “amorphous” whenever used, means the amorphous form having less than 5% of the crystalline form. In particular the amorphous form of the present invention contains less than 2%, less than 0.5% or not in detectable amount of the crystalline form when measured by x-ray powder diffraction (XRD).

As used herein the term “suspension” whenever used, may be interchangeable with “slurry” and includes a heterogeneous mixture wherein complete dissolution does not occur or heating the suspension or slurry to obtain a homogenous mixture where complete or partial dissolution occurs at an elevated temperature or ambient temperature.

All ranges recited herein include the endpoints, including those that recite a range “between” two values. Terms such as “about”, “generally”, and “substantially,” are to be construed as modifying a term or value such that it is not an absolute. This includes, at very least, the degree of expected experimental error, technique error and instrument error for a given technique used to measure a value.

The product obtained by the process of the present invention may be further dried to achieve the desired levels of moisture content and/or LOD value. For example, the product may be dried in a tray drier, dried under vacuum and/or in a Fluid Bed Drier.

As used herein, “Particle Size Distribution (PSD)” means the cumulative volume size distribution of equivalent spherical diameters as determined by laser diffraction in Malvern Master Sizer 2000 equipment or its equivalent.

The important characteristics of the PSD are the (D₉₀), which is the size, in microns, below which 90% of the particles by volume are found, and the (D₅₀), which is the size, in microns, below which 50% of the particles by volume are found. Thus, a D₉₀ or d(0.9) of 450 μm or less means the 90 volume-percent of the particles in a composition have a diameter less than 450 μm.

The term “pharmaceutically acceptable” means that which is useful in preparing a pharmaceutical composition that is generally non-toxic and is not biologically undesirable, and includes that which is acceptable for veterinary use and/or human pharmaceutical use.

The term “pharmaceutical composition” is intended to encompass a drug product including the active ingredient(s) lomitapide free base or lomitapide mesylate, pharmaceutically acceptable excipients that make up the carrier, as well as any product which results, directly or indirectly, from combination, complexation or aggregation of any two or more of the ingredients. Accordingly, the pharmaceutical compositions encompass any composition made by admixing the active ingredient, active ingredient dispersion or composite, additional active ingredient(s), and pharmaceutically acceptable excipients.

The solutions prior to any solid formation may, optionally, be filtered to remove any undissolved solids and/or impurities prior to removal of solvent.

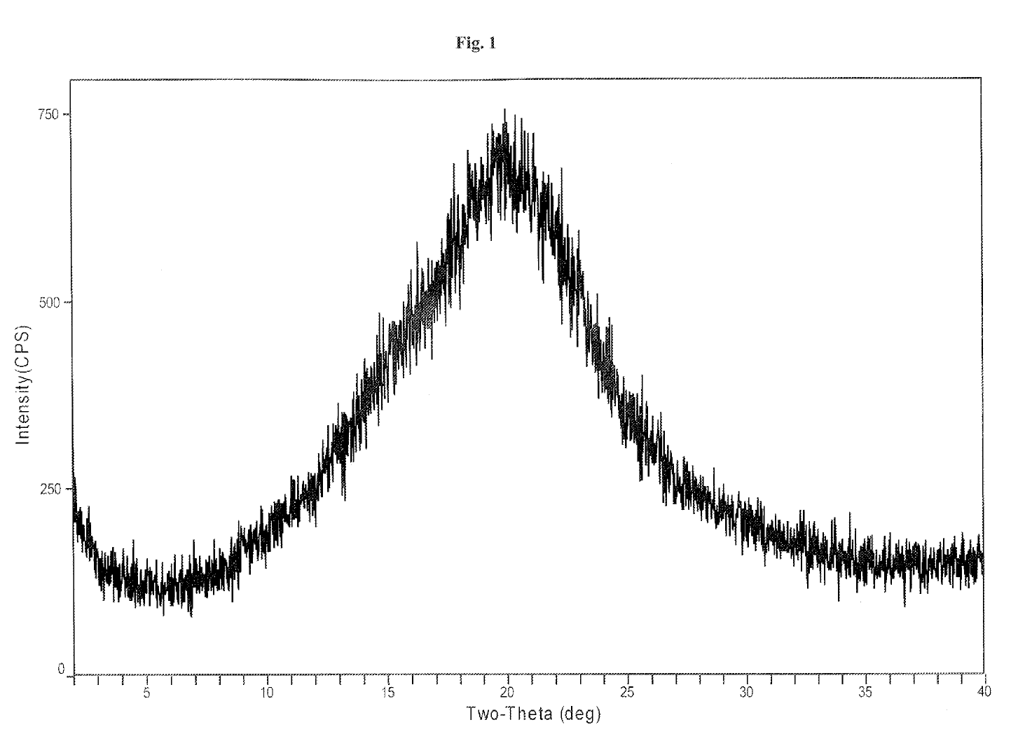

In one general aspect, there is provided lomitapide mesylate in solid amorphous form. In general, the lomitapide mesylate in solid amorphous form is characterized by x-ray powder diffraction pattern substantially as shown in FIG. 1.

In general, the lomitapide mesylate in solid amorphous form contains less than 5% of the crystalline form, preferably less than 2%, more preferably less than 0.5% and most preferably in not detectable amount of crystalline form when measured by x-ray powder diffraction.

In general, the lomitapide mesylate in solid amorphous form having less than 0.5% residual solvents when measured by GC. In particular, the lomitapide mesylate in solid amorphous form having less than 0.3%, particularly less than 0.1% of residual solvents when measured by GC.

In general, the lomitapide mesylate in solid amorphous form having a moisture content less than 5% wt/wt, particularly less than 2.5% wt/wt, more particularly less than 1% wt/wt and most particularly less than 0.5% wt/wt.

In general, the lomitapide mesylate in solid amorphous form of present invention is stable during drying and storage.

As used herein, the term “stable” includes either: amorphous lomitapide mesylate which is stable for a period of at least three months after exposure to a relative humidity of 75% at 40° C. or to a relative humidity of 60% at 25° C., and does not change to any crystalline form and contains less than about 0.5% (wt/wt) total impurities.

In another general aspect, there is provided a process for the preparation of an amorphous form of lomitapide mesylate, the process comprising;
(a) providing a solution of lomitapide mesylate in one or more solvents; and
(b) obtaining the amorphous form of lomitapide mesylate by the removal of the solvents.

In another general aspect, there is provided a process for the preparation of an amorphous form of lomitapide mesylate, the process comprising:
(a) providing a solution of lomitapide free base in one or more solvents;
(b) adding methane sulfonic acid to the solution; and
(c) obtaining the amorphous form of lomitapide mesylate by the removal of the solvents or by adding one or more of anti-solvents to the solution.

In general, the solution of lomitapide mesylate may be provided as a reaction mixture containing lomitapide mesylate in course of its synthesis and/or purification or by dissolving lomitapide mesylate in one or more solvents. Alternatively, lomitapide free base and methane sulfonic acid may be dissolved in same or different solvents and then contacted with each other to obtain the lomitapide mesylate solution in situ or directly adding methanesulfonic acid to lomitapide free base with stirring to give lomitapide mesylate which may be further dissolved in one or more solvents.

In general, the solvent comprises one or more of alcohols selected from methanol, ethanol, n-propanol, isopropanol (IPA), and n-butanol; esters selected from ethyl acetate, propyl acetate, isopropyl acetate, t-butyl acetate, and isobutyl acetate; ketones selected from acetone, methyl ethyl ketone, and methyl isobutyl ketone; halogenated hydrocarbons is selected from methylene dichloride, ethylene dichloride, carbon tetrachloride and chlorobenzene; polar aprotic solvent is selected from dimethylformamide, dimethylsulfoxide, and N-methylpyrrolidone; tetrahydrofuran, 2-methyl tetrahydrofuran, dioxane, acetonitrile, or mixtures thereof.

In general, the solvent from the solution of lomitapide mesylate may be removed by one or more techniques selected from distillation, distillation under vacuum, evaporation, evaporation by rotational distillation, spray drying, agitated thin film drying (“ATFD”), freeze drying (lyophilization), filtration, decantation, and centrifugation to obtain amorphous form of lomitapide mesylate. The solvent may also be removed, optionally, at reduced pressure and/or elevated temperature.

In general, freeze drying (lyophilization) may be performed by freezing a solution of lomitapide mesylate at low temperatures and reducing the pressure to remove the solvent from the frozen solution of lomitapide mesylate. Temperatures that may be required to freeze the solution, depending on the solvent chosen to make the solution of lomitapide mesylate may range from about −70° C. to about 10° C.

In another general aspect, there is provided a process for preparation of amorphous form of lomitapide mesylate comprises spray drying a solution of lomitapide mesylate in one or more solvents. In general, the process comprising the spray drying of a feed stock.

In general, the feed stock which is prepared as discussed herein below. The preparation of the feed stock comprises use of lomitapide mesylate prepared by process of the present invention. In particular, the spray drying of lomitapide mesylate may be carried out by maintaining the inlet temperature in the range of 35° C.-80° C., nitrogen pressure of 2-4 kg/cm², maintaining the outlet temperature in the range of 30° C. to 60° C., at a feed rate of 15% to 20% and maintaining the vacuum at 30-120 mm of Hg using JISL Mini LSD-48 or LU-222 advanced model (twin cyclone) type spray driers.

In general, the solution, suspension or slurry comprising lomitapide mesylate may be spray-dried to obtain an amorphous form of lomitapide mesylate.

In general, the solution of lomitapide mesylate may be evaporated by rotational distillation device to obtain an amorphous form of lomitapide mesylate.

In another general aspect, the amorphous form of lomitapide mesylate may also be obtained by adding one or more anti-solvents to the solution of lomitapide mesylate in one or more solvents or by adding one or more anti-solvents to the residue obtained by partial or complete removal of the solvents. The anti-solvents may be added to the solution of lomitapide mesylate or the solution may be added to one or more anti-solvents.

In general, the anti-solvent comprises one or more of diethylether, diisopropyether, methyl tert-butyl ether, tetrahydrofuran, 1,4-dioxane, hexane, heptane, octane, cyclohexane, toluene, xylene and ethylbenzene.

In general, the amorphous form of lomitapide mesylate obtained thereby may be recovered by removal of solvent by one or more methods selected from decantation, filtration or centrifugation.

In another general aspect, there is provided a process for the preparation of amorphous form of lomitapide mesylate, the process comprising;
(a) providing a solution of lomitapide in one or more of first organic solvent;
(b) providing a solution of methanesulfonic acid in one or more of second organic solvent; and
(c) contacting the solutions of steps a) and b) to obtain amorphous form of lomitapide mesylate.

In, general, the first and the second organic solvents may be same or different and can be selected from the organic solvents and anti-solvents described herein above.

In general, the first organic solvent may be selected from acetone and methanol or mixtures thereof; and the second organic solvent may be selected from ethyl acetate and water or mixtures thereof.

In another general aspect, there is provided an amorphous solid dispersion of lomitapide mesylate and a pharmaceutically acceptable carrier.

The term “solid dispersion” means any solid composition having at least two components. In certain embodiments, a solid dispersion as disclosed herein includes the active ingredient, lomitapide mesylate, dispersed among at least one pharmaceutically acceptable carrier.

In general, the pharmaceutically acceptable carrier is a polymer. The polymer may be a non-ionic polymer or an ionic polymer comprising one or more of hydroxypropylmethyl cellulose acetate succinate (HPMC-AS), hydroxypropylmethyl cellulose (HPMC), methacrylic acid copolymers and polyvinylpyrrolidone (PVP). In particular, hydroxypropylmethyl cellulose or its acetate succinate may be used.

In another general aspect, there is provided a process for the preparation of a lomitapide mesylate, the process comprising reacting lomitapide free base with methane sulfonic acid.

In general, the lomitapide free base may be reacted with methane sulfonic acid in presence of one or more solvents. The solvents comprises one or more of C₁₋₄alcohols, C₂₋₆esters, ketones, halogenated hydrocarbon, polar aprotic solvent, tetrahydrofuran, 2-methyltetrahydrofuran, dioxane or mixtures thereof.

In general, the C₁₋₄alcohol is selected from methanol, ethanol, n-propanol, isopropanol (IPA), and n-butanol; the C₂₋₆ester is selected from ethyl acetate, propyl acetate, isopropyl acetate, t-butyl acetate, and isobutyl acetate; the ketone is selected from acetone, methyl ethyl ketone, and methyl isobutyl ketone; the halogenated hydrocarbon is selected from methylene dichloride, ethylene dichloride, carbon tetrachloride and chlorobenzene; the polar aprotic solvent is selected from dimethylformamide, dimethylsulfoxide, and N-methylpyrrolidone, or mixture thereof.

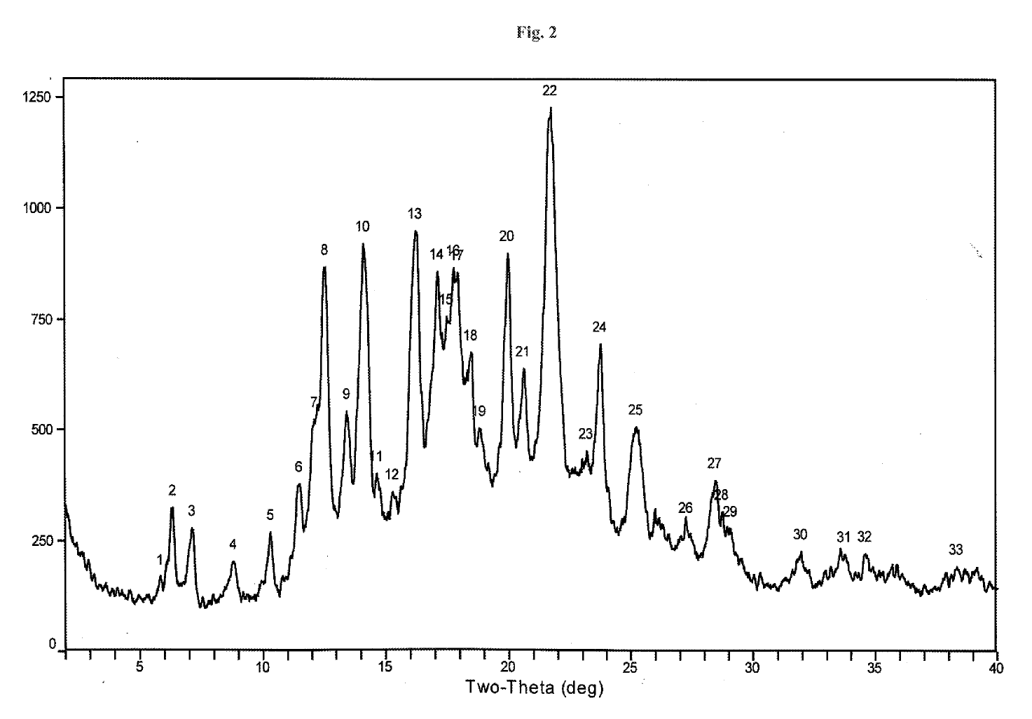

In another general aspect, there is provided a crystalline Form-A of lomitapide mesylate (optionally and interchangeably “The Form A” herein after). In general, the Form-A of lomitapide mesylate is characterized by x-ray powder diffraction pattern having characteristic peaks expressed in terms of 2θ at about 6.3°, 12.6°, 14.1°, 16.2°, 21.7° and 23.7°±0.2° (2θ) and x-ray powder diffraction pattern substantially as shown in FIG. 2.

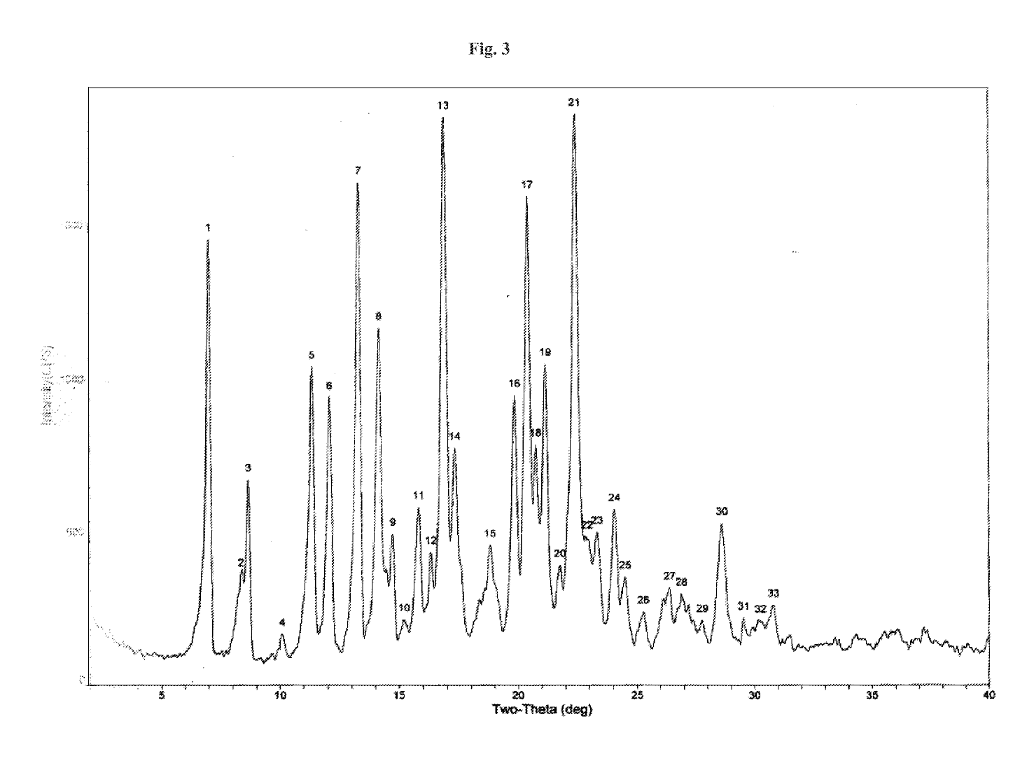

In another general aspect, there is provided a crystalline Form-B of lomitapide mesylate (optionally and interchangeably “The Form B” herein after). In general, the Form-B of lomitapide mesylate is characterized by x-ray powder diffraction pattern having characteristic peaks expressed in terms of 2θ at about 7.0°, 11.3°, 12.1°, 13.3°, 16.9°, 22.4° and 28.6°±0.2° (2θ) and x-ray powder diffraction pattern substantially as shown in FIG. 3.

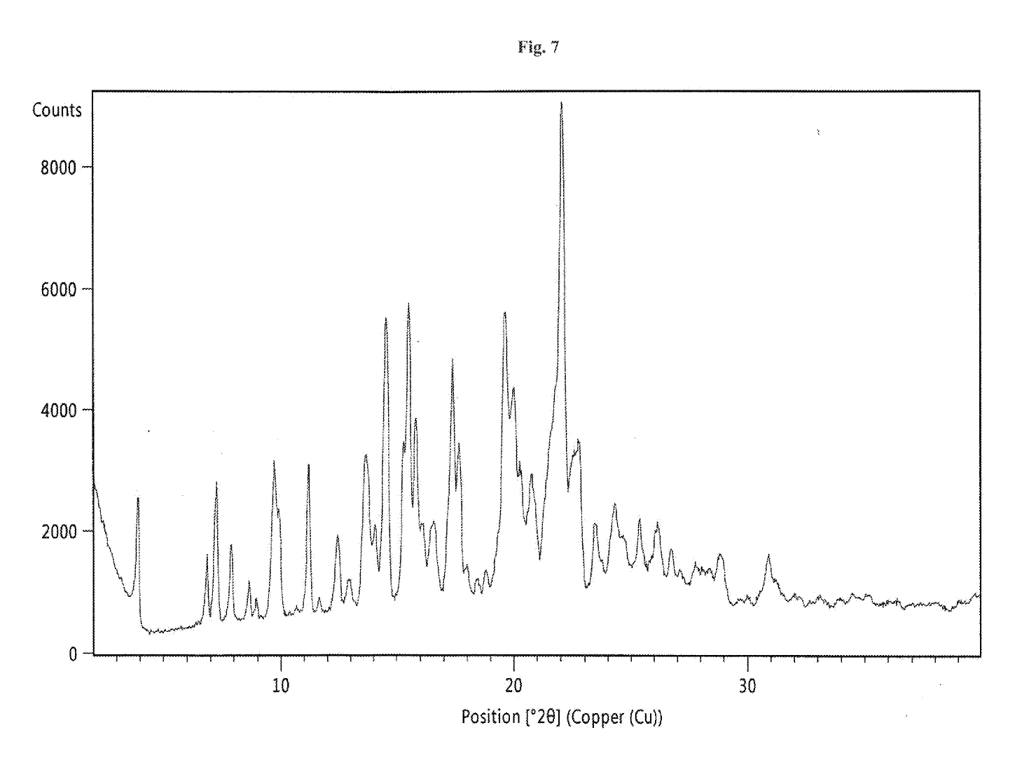

In another general aspect, there is provided a crystalline Form-C of lomitapide mesylate (optionally and interchangeably “The Form C” herein after). In general, the Form-C is characterized by x-ray powder diffraction pattern having characteristic peaks expressed in terms of 2θ at about 3.9, 9.9, 15.5, 19.5 and 22.0°±0.2° (2θ) and x-ray powder diffraction pattern substantially as shown in FIG. 7.

The Form-C is further characterized by x-ray powder diffraction pattern having additional peaks expressed in terms of 2θ at 7.2, 11.2, 13.6, 14.6, 15.8, 17.4, 17.7, 20.0 and 22.8°±0.2°.

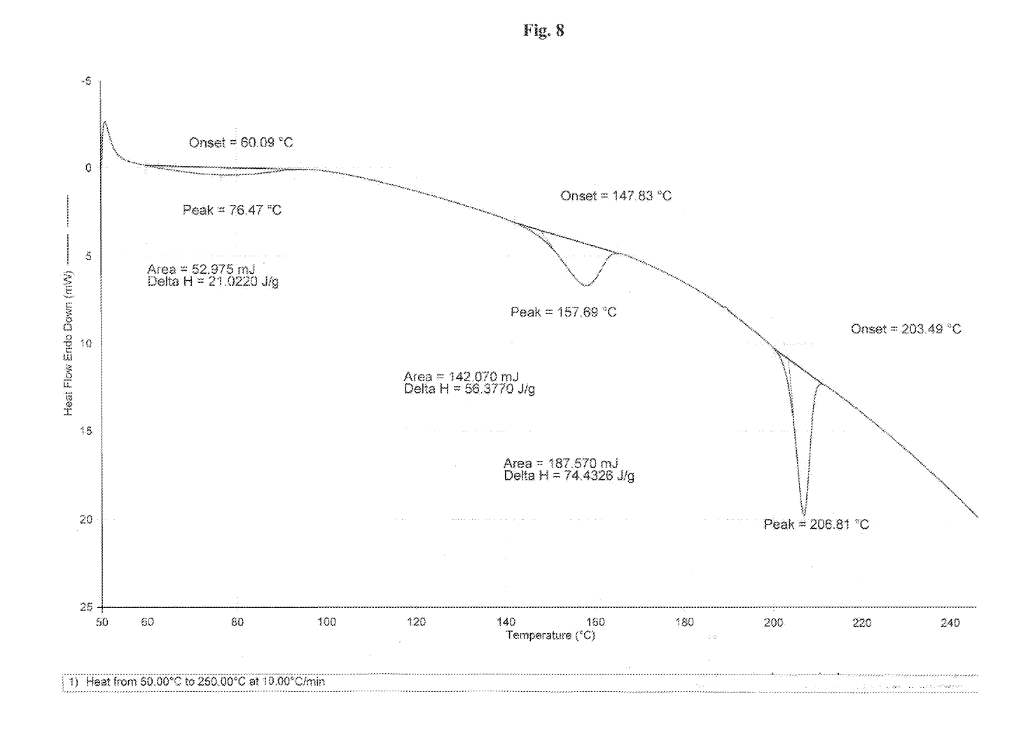

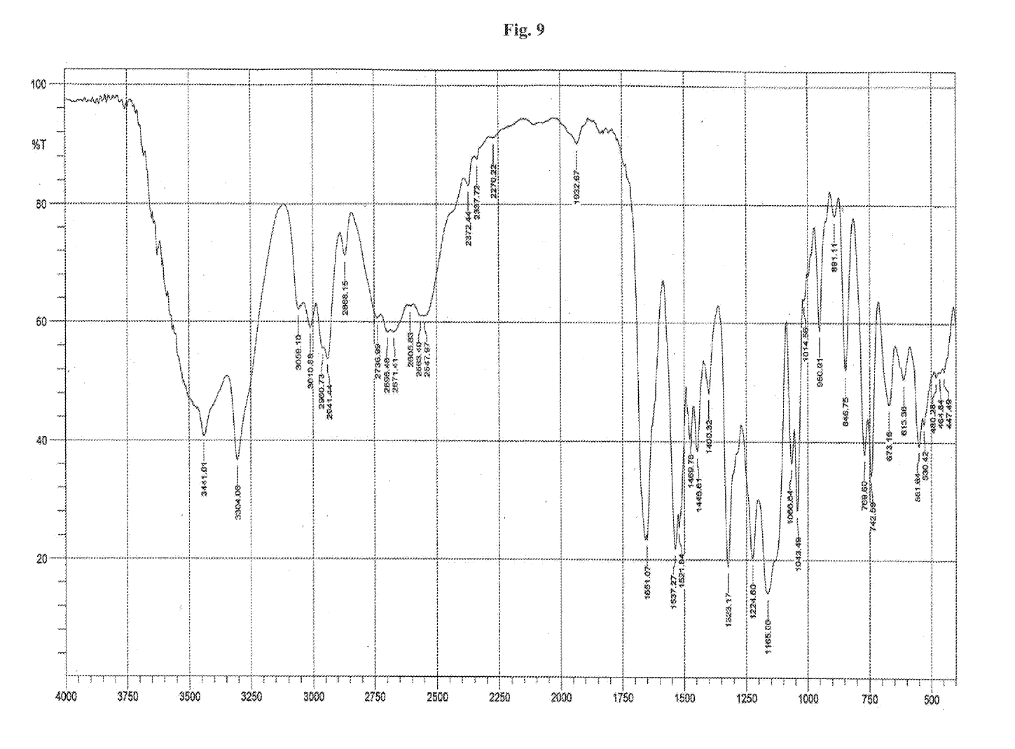

The Form-C is further characterised by differential scanning calorimetery (DSC) as depicted in FIG. 8 and FT-IR spectrum as depicted in FIG. 9.

In another general aspect, there is provided a process for the preparation of a crystalline form of lomitapide mesylate, the process comprising:
(a) providing a solution of lomitapide mesylate in one or more solvents; and
(b) obtaining the crystalline form of lomitapide mesylate by removal of the solvents.

In general, the solution of lomitapide mesylate may be provided as a reaction mixture containing lomitapide mesylate in course of its synthesis and/or purification or by dissolving lomitapide mesylate in one or more solvents. Alternatively, lomitapide free base and methane sulfonic acid may be dissolved in same or different solvents and then contacted with each other to obtain the lomitapide mesylate solution in situ or directly adding methanesulfonic acid to lomitapide free base with stirring to give lomitapide mesylate which may be further dissolved in solvent.

In general, the solvent comprises one or more of alcohols, esters, ketones, halogenated hydrocarbons, polar aprotic solvent, tetrahydrofuran, 2-methyl tetrahydrofuran, dioxane, acetonitrile, or mixtures thereof.

In general, the alcohol is selected from methanol, ethanol, n-propanol, isopropanol (IPA), and n-butanol; the ester is selected from ethyl acetate, propyl acetate, isopropyl acetate, t-butyl acetate, and isobutyl acetate; the ketone is selected from acetone, methyl ethyl ketone, and methyl isobutyl ketone; the halogenated hydrocarbon is selected from methylene dichloride, ethylene dichloride, carbon tetrachloride and chlorobenzene; the polar aprotic solvent is selected from dimethylformamide, dimethylsulfoxide, and N-methylpyrrolidone, acetonitrile, or mixture thereof.

In general, the solution of lomitapide mesylate in one or more solvents may be heated from about 35° C. to about reflux temperature of the solvent to obtain the clear solution. In particular, the solution may be heated from about 35° C. to about 120° C. The solution is maintained for sufficient time at reflux temperature. In particular from about 30 minutes to about 48 hours.

The solution is cooled to ambient temperature. In particular, the solution to be cooled to about less than 35° C. The solution is further gradually cooled to 0° C. to 10° C.

In general, the solvent from the cooled solution of lomitapide mesylate may be removed by one or more techniques selected by filtration, decantation, and/or centrifugation to obtain crystalline form of lomitapide mesylate. The solvent may also be removed, optionally, at reduced pressure and/or elevated temperature.

In another general aspect, there is provided a process for the preparation of a crystalline Form-A of lomitapide mesylate, the process comprising:
(a) providing a solution of lomitapide mesylate in one or more ester solvent;
(b) heating the solution; and
(c) obtaining the crystalline Form-A of lomitapide mesylate by the removal of the solvent.

In general, the ester solvent comprises one or more of ethyl acetate, propyl acetate, ispropyl acetate, butyl acetate and isobutyl acetate.

In another general aspect, there is provided a process for the preparation of a crystalline Form-B of lomitapide mesylate, the process comprising:
(a) providing a solution of lomitapide mesylate in a nitrile solvent;
(b) heating the solution, and
(c) obtaining the crystalline Form-B of lomitapide mesylate by the removal of the solvent.

In general, the nitrile solvent comprises one or more of acetonitrile and propionitrile.

In another general aspect, there is provided a process for the preparation of crystalline Form-C of lomitapide mesylate, the process comprising:
(a) providing a solution of lomitapide mesylate in a solvent selected from alcoholic solvents, halogenated solvents, hydrocarbons or mixtures thereof; and
(b) obtaining the crystalline Form-C of lomitapide mesylate by the removal of the solvent.

In general, the alcoholic solvent is selected from methanol, ethanol, n-propanol, isopropanol (IPA), and n-butanol; halogenated solvent is selected from methylene dichloride, carbon tetrachloride and chloroform; hydrocarbon is selected from n-hexane, n-heptane, cyclohexane, toluene and xylene. In particular, the alcohol is isopropanol, halogenated solvent is methylene dichloride and hydrocarbon is n-heptane.

In general, the Form-C is obtained by crystallizing lomitapide mesylate from an alcoholic solvent or mixture of a halogenated solvent and a hydrocarbon; more preferably the Form-C is obtained by crystallizing lomitapide mesylate from isopropanol or methylene dichloride/n-heptane.

In general, the solution of lomitapide mesylate in one or more solvent(s) may be heated from 35° C. to reflux temperature of the solvent to obtain the clear solution. In particular, the solution may be heated from 35° C. to 80° C. The solution is maintained for sufficient time at reflux temperature. In particular, the solution is maintained for, from less than 1 minute to more than 24 hours. The solution is than cooled to ambient temperature. In particular, the solution is cooled to less than 35° C. The solution may further be cooled to less than 20° C.

In general, the solvent from the cooled solution of lomitapide mesylate may be removed by one or more techniques selected by filtration, decantation, and/or centrifugation to obtain the Form-C of lomitapide mesylate. The solvent may also be removed, optionally, at reduced pressure and/or elevated temperature.

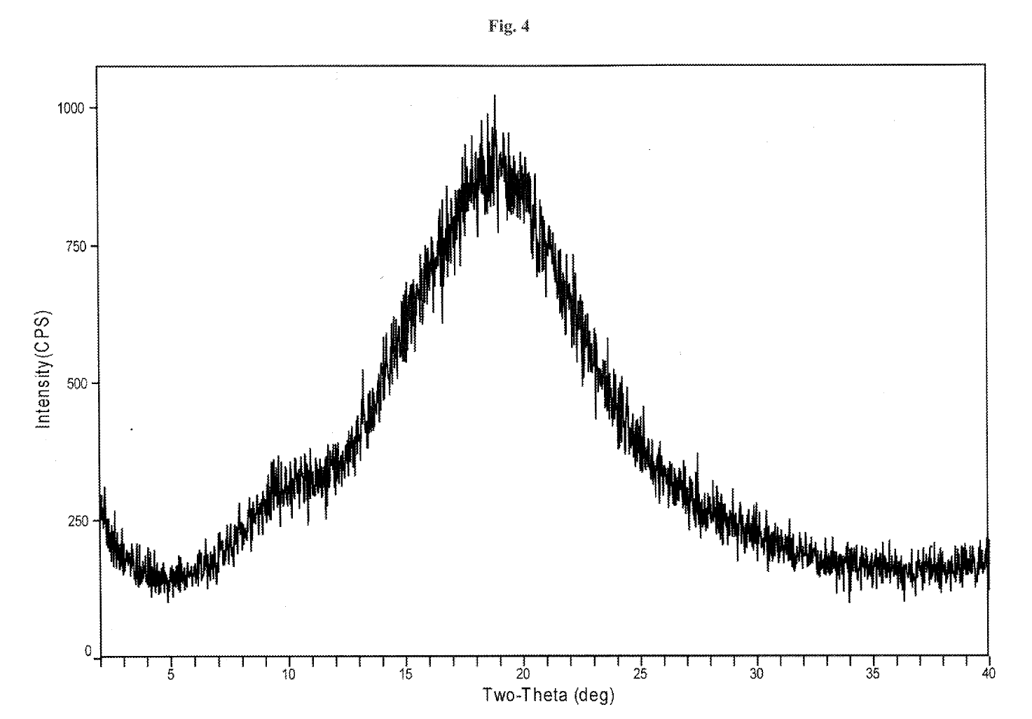

In another general aspect, there is provided lomitapide free base in solid amorphous form. In general, lomitapide free base in solid amorphous form is characterized by x-ray powder diffraction pattern substantially as shown in FIG. 4.

In another general aspect, there is provided a process for the preparation of an amorphous form of lomitapide free base, the process comprising:
(a) providing a solution of lomitapide free base in one or more solvents; and
(b) obtaining the amorphous form of lomitapide free base by removal of the solvents or by adding one or more of anti-solvents to the solution.

In general, the solvent comprises one or more of alcohols, esters, ketones, ethers, halogenated hydrocarbons, aromatic hydrocarbons, polar aprotic solvent or mixtures thereof.

In general, the alcohol is selected from methanol, ethanol, n-propanol, isopropanol (IPA), and n-butanol; the ester is selected from ethyl acetate, propyl acetate, isopropyl acetate, t-butyl acetate, and isobutyl acetate; the ketones is selected from acetone, methyl ethyl ketone, and methyl isobutyl ketone; the ether is selected from diethylether, diisopropyl ether, methyl tert-butyl ether, tetrahydrofuran, 2-methyltetrahydrofura, 1,4-doxane, the halogenated hydrocarbon is selected from methylene dichloride, ethylene dichloride, carbon tetrachloride and chlorobenzene; the aromatic hydrocarbon is selected from toluene, xylene, chlorobenzene and ethylbenzene, the polar aprotic solvent is selected from dimethylformamide, dimethylsulfoxide, and N-methylpyrrolidone, or mixture thereof.

In general, the solution of lomitapide free base may be heated from about 35° C. to about reflux temperature of the solvent. In particular, the solution is heated from about 35° C. to about 150° C.

In general, the solvent from the solution of lomitapide free base may be removed by one or more techniques selected from distillation, distillation under vacuum, evaporation, evaporation by rotational distillation, spray drying, agitated thin film drying (“ATFD”), freeze drying (lyophilization), filtration, decantation, and centrifugation to obtain amorphous form of lomitapide free base. The solvent may also be removed, optionally, at reduced pressure and/or elevated temperature.

In general, freeze drying (lyophilization) may be performed by freezing a solution of lomitapide free base at low temperatures and reducing the pressure to remove the solvent from the frozen solution of lomitapide mesylate. Temperatures that may be required to freeze the solution, depending on the solvent chosen to make the solution of lomitapide free base may range from about −70° C. to about 10° C.

In another general aspect, there is provided a process for the preparation of amorphous form of lomitapide free base comprising spray drying a solution of lomitapide free base in one or more solvent(s). In general, the process involves spray drying of the feed stock.

In general, the preferred aspect of the invention involves spray drying of feed stock which is prepared as discussed below, wherein any solid forms of lomitapide free base is used. In particular, the spray drying of lomitapide free base may be carried out by maintaining the inlet temperature in the range of 35° C.-80° C., nitrogen pressure of 2-4 kg/cm², maintaining the outlet temperature in the range of 30° C. to 60° C., at a feed rate of 15% to 20% and maintaining the vacuum at 30-120 mm of Hg using JISL Mini LSD-48 or LU-222 advanced model (twin cyclone) type spray driers.

In general, the solution, suspension or slurry comprising lomitapide free base may be spray-dried to get amorphous form of lomitapide free base.

In general, the solution of lomitapide free base may be evaporated using a rotational distillation device to obtain an amorphous form of lomitapide free base.

In another general aspect, the amorphous form of lomitapide free base may also be obtained by adding one or more anti-solvents to the solution in one or more solvent or by adding one or more anti-solvents to the residue obtained by partial or complete removal of the solvents. The anti-solvents may be added to the solution of lomitapide free base or the solution may be added to one or more anti-solvents.

In general, the anti-solvent comprises one or more of water, hexane, heptane, octane, and cyclohexane. The anti-solvent may be added to the pre-cooled solution of lomitapide free base in one or more solvents. In particular, the pre-cooled solution may be cooled up to less than 25° C. more particularly up to less than 10° C.

In general, the amorphous form of lomitapide free base obtained thereby may be recovered by the removal of the solvent by one or more methods selected from decantation, filtration or centrifugation.

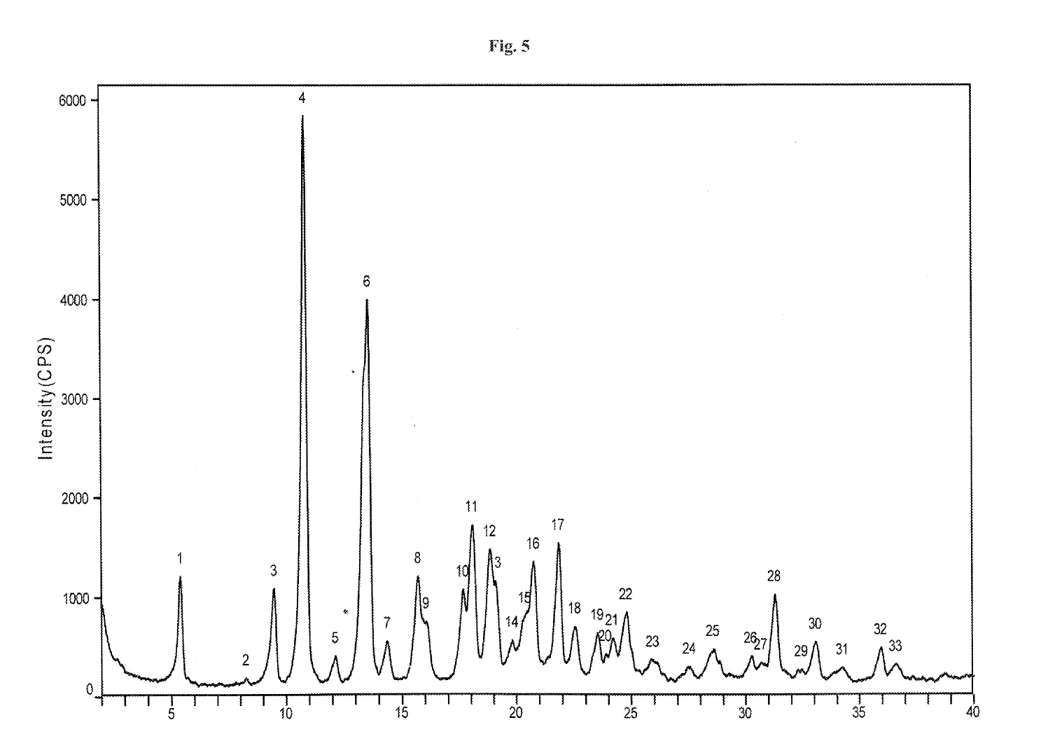

In another general aspect, there is provided a crystalline Form-I of lomitapide free base. In general, the crystalline Form-I of lomitapide free base is characterized by x-ray powder diffraction pattern having characteristic peaks expressed in terms of 2θ at about 5.4°, 10.8°, 13.6°, 21.8°, and 31.3°±0.2° (2θ) and x-ray powder diffraction pattern substantially as shown in FIG. 5.

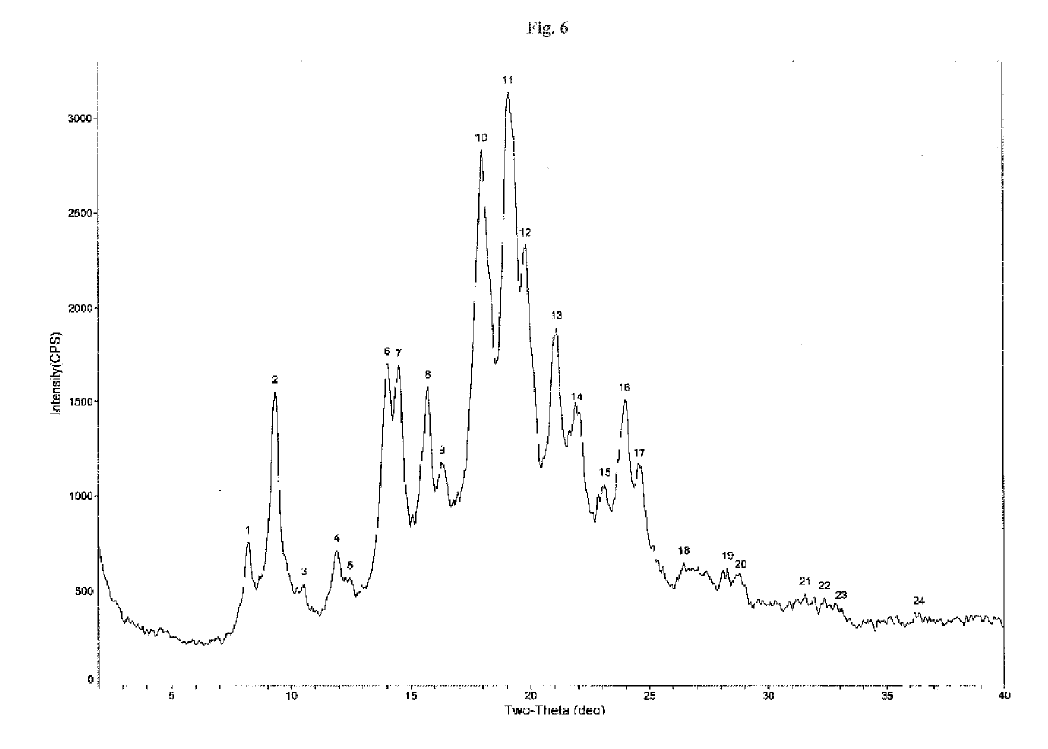

In another general aspect, there is provided a crystalline Form-II of lomitapide free base. In general, the crystalline Form-II of lomitapide free base is characterized by x-ray powder diffraction pattern having characteristic peaks expressed in terms of 2θ at about 9.3°, 18.0°, 19.1°, 21.1°, and 23.9°±0.20 (2θ) and x-ray powder diffraction pattern substantially as shown in FIG. 6.

In another general aspect, there is provided a process for the preparation of a crystalline Form-I of lomitapide free base, the process comprising:
(a) providing a solution of lomitapide free base in one or more solvents; and
(b) obtaining the crystalline Form-I of lomitapide free base by the removal of the solvents.

In general, the solvent comprises one or more of alcohols selected from methanol, ethanol, n-propanol, isopropanol (IPA), and n-butanol; esters selected from ethyl acetate, propyl acetate, isopropyl acetate, t-butyl acetate, and isobutyl acetate; ketones selected from acetone, methyl ethyl ketone, and methyl isobutyl ketone; ethers selected from diethyl ether, diisopropyl ether, methyl tert-butyl ether, tetrahydrofuran, 2-methyltetrahydrofuran, and 1,4-doxane; halogenated hydrocarbons selected from methylene dichloride, ethylene dichloride, carbon tetrachloride and chlorobenzene; polar aprotic solvent selected from dimethylformamide, dimethylsulfoxide, and N-methylpyrrolidone, or mixtures thereof.

In another general aspect, there is provided a process for the preparation of a crystalline Form-II of lomitapide free base, the process comprising:
(a) providing a solution of lomitapide free base in one or more hydrocarbon solvents; and
(b) obtaining the crystalline Form-II of lomitapide free base by the removal of the solvents.

In general, the hydrocarbon solvent is selected from one or more of toluene, xylene or ethylbenzene.

In another general aspect, there is provided a process for the preparation of crystalline Form-I of lomitapide free base, the process comprising:
(a) providing a solution of lomitapide free base in one or more solvents;
(b) heating the solution; and
(c) obtaining the crystalline form of lomitapide free base by adding one or more anti-solvents.

In general, the solution of lomitapide free base in one or more solvent(s) may be heated from about 35° C. to about reflux temperature of the solvent to obtain the clear solution. In particular, the solution may be heated from about 35° C. to about 120° C. The solution is maintained for sufficient time at reflux temperature. In particular from about 30 minutes to about 48 hours.

The solution is cooled to ambient temperature. In particular, the solution to be cooled to about less than 35° C. The solution is further gradually cooled to 0° C. to 10° C.

In general, the solvent from the cooled solution of lomitapide free base may be removed after by one or more techniques selected by filtration, decantation, and/or centrifugation to obtain crystalline form of lomitapide mesylate. The solvent may also be removed, optionally, at reduced pressure and/or elevated temperature.

In another general aspect, the crystalline form of lomitapide free base may also be obtained by adding an anti-solvent to the solution of lomitapide free base in one or more solvent or by adding anti-solvent to the residue obtained by partial or complete removal of the solvent. The anti-solvent may be added to the solution of lomitapide free base or the solution may be added to the anti-solvent.

In general, the anti-solvent is selected from one or more of water, hexane, heptane, octane, cyclohexane, toluene, xylene and ethylbenzene.

The anti-solvent may be added to the pre-cooled solution of lomitapide free base or heated solution of lomitapide free base in one or more solvents. In particular, the pre-cooled solution may be cooled up to less than 25° C., more particularly up to less than 10° C. In particular, the heated solution may be from about 50° C. to about 120° C., more particularly the heated solution may be from about 50° C. to about 100° C.

In general, the crystalline form of lomitapide free base obtained thereby may be recovered by the removal of the solvent by one or more methods selected from decantation, filtration or centrifugation.

In another general aspect, there is provided a pharmaceutical composition comprising an amorphous form of lomitapide mesylate and pharmaceutically acceptable carriers, diluents and excipients.

In another general aspect, there is provided a pharmaceutical composition comprising a crystalline Form-A of lomitapide mesylate and pharmaceutically acceptable carriers, diluents and excipients.

In another general aspect, there is provided a pharmaceutical composition comprising a crystalline Form-B of lomitapide mesylate and pharmaceutically acceptable carriers, diluents and excipients.

In another general aspect, there is provided a pharmaceutical composition comprising the Form-C of lomitapide mesylate and pharmaceutically acceptable carriers, diluents and excipients.

In another general aspect, there is provided a pharmaceutical composition comprising a crystalline form of lomitapide free base and pharmaceutically acceptable carriers, diluents and excipients.

In another general aspect, there is provided a pharmaceutical composition comprising an amorphous form of lomitapide free base and pharmaceutically acceptable carriers, diluents and excipients.

In another general aspect, there is provided an amorphous form of lomitapide mesylate having a purity of about 98% or more, as measured by an area percentage by HPLC. In particular, the amorphous form of lomitapide mesylate is having a purity of about 99% or more, particularly of about 99.5% or more, more particular of about 99.8% or more, when measured by an area percentage by HPLC.

In another general aspect, there is provided a crystalline form of lomitapide mesylate having a purity of about 98% or more, when measured by an area percentage by HPLC. In particular, the crystalline form of lomitapide mesylate is having a purity of about 99% or more, particularly of about 99.5% or more, more particular of about 99.8% or more, when measured by an area percentage by HPLC.

In general, the amorphous form of lomitapide mesylate is having particle size distribution as characterized by 90% particles having particle size (D₉₀) of about 250 μm or less, 50% particles having particle size (D₅₀) of about 100 μm or less, and 10% particles having particle size (D₁₀) of about 50 μm or less.

In further aspect, the amorphous form of lomitapide mesylate may be micronized to achieve the better particle size distribution in order to make suitable Formulation.

In general, the crystalline form of lomitapide mesylate is having particle size distribution as characterized by 90% particles having particle size (D₉₀) of about 250 μm or less, 50% particles having particle size (D₅₀) of about 100 μm or less, and 10% particles having particle size (D₁₀) of about 50 μm or less.

In further aspect, the crystalline form of lomitapide mesylate may be micronized to achieve the better particle size distribution in order to make suitable Formulation.

In general, the starting compound lomitapide may be prepared by the process disclosed in U.S. Pat. No. 5,712,279, which is incorporated herein as reference in its entirety.

The embodiments of the present invention are further described using specific examples herein after. The examples are provided for better understanding of certain embodiments of the invention and not, in any way, to limit the scope thereof. Plausible modifications and equivalents apparent to those skilled in the art using the teachings of the present description and the general art in the field of the invention shall also form the part of this specification and are intended to be included within the scope of it.

### EXAMPLES

### Example-1

### Preparation of Lomitapide Mesylate

In a 250 mL round bottom flask, equipped with a mechanical stirrer, thermometer and an addition funnel, 10 g lomitapide and 20 mL methanol were added and stirred to obtain a solution. 1.5 g methane sulfonic acid dissolved in 20 mL water was added slowly to the above solution under stirring. The reaction mixture was stirred till maximum salt formation was achieved. 50 mL water was added to the mixture, stirred for 15-20 min, filtered and washed with water. The product was dried further to obtain lomitapide mesylate.

### Example-2

### Preparation of Amorphous Form of Lomitapide Mesylate

10 g lomitapide mesylate, 50 mL acetone and 150 mL ethyl acetate were heated in a 500 mL round bottom flask, equipped with a mechanical stirrer, thermometer and an addition funnel at 50-55° C. and stirred to obtain clear solution. The solution was subjected to spray drying in JISL Mini spray drier LSD-48 with feed pump running at 30-35 rpm, inlet temperature 50-55° C., out let temperature 45-50° C., aspiration rate 1200-1300 rpm, hot air supply 1.8-2.2 Kg/cm² and vacuum for conveying the dry product 80 mmHg. The product was collected from cyclone and characterized to an amorphous form by x-ray powder diffraction. The product was further dried to obtain the amorphous form of lomitapide mesylate.

### Example-3

### Preparation of Amorphous Solid Dispersion of Lomitapide Mesylate

Lomitapide mesylate (10 g), hydroxypropylmethyl cellulose (10 g) acetone (100 mL) and ethyl acetate (250 mL) were heated to 50-55° C. and stirred to obtain clear solution followed by spray drying in JISL Mini spray drier LSD-48 as per parameters described in example-2. The product collected from cyclone was further dried to obtain amorphous solid dispersion of lomitapide mesylate.

### Example-4

### Preparation of Amorphous Form of Lomitapide Mesylate

In a 500 mL round bottom flask, equipped with a mechanical stirrer, thermometer and an addition funnel, 10 g lomitapide free base and 50 mL acetone were taken at 35° C. 1.45 g methane sulfonic acid was added to the reaction mixture and stirred for 1 hour. The acetone was distilled under vacuum completely below 40° C. The reaction mixture was cooled to obtain amorphous form of lomitapide mesylate.

### Example-5

### Preparation of Amorphous Form of Lomitapide Mesylate

In a 500 mL round bottom flask, equipped with a mechanical stirrer, thermometer and an addition funnel, 10 g lomitapide free base and 50 mL acetone were taken at 35° C. 1.45 g methane sulfonic acid was added to the reaction mixture and stirred for 1 hour. The acetone was distilled under vacuum completely below 40° C. 50 mL diisopropyl ether was stirred for 30 min at 55° C. and cooled to 35° C. The product is filtered and washed with diisopropyl ether. The product was dried at 40° (C to 45° C. and cooled to 30° C. to obtain amorphous form of lomitapide mesylate.

### Example-6

### Preparation of Amorphous Form of Lomitapide Free Base

In a 500 mL round bottom flask, equipped with a mechanical stirrer, thermometer and an addition funnel, 10 g lomitapide base and 100 mL toluene were heated at 60° C. to 65° C. to obtain clear solution. The clear solution was distilled completely to remove toluene below 60° C. and cooled to 35° C. to obtain amorphous form of lomitapide free base.

### Example-7

### Preparation of Amorphous Form of Lomitapide Free Base

In a 500 mL round bottom flask, equipped with a mechanical stirrer, thermometer and an addition funnel, 10 g lomitapide base and 20 mL acetone were warmed at 35° C. The reaction mixture was cooled to 0° C. to 5° C. and 200 mL water was added. The reaction mixture was stirred for 30 min. The precipitated product was filtered and washed with water. The product was dried at 55° C. to 60° C. in hot air oven and cooled to 30° C. to 35° C. to obtain amorphous form of lomitapide free base.

### Example-8

### Preparation of Crystalline Form-I of Lomitapide Free Base

In a 500 mL round bottom flask, equipped with a mechanical stirrer, thermometer and an addition funnel, 10 g lomitapide base and 50 mL diisopropylether were heated at 65° C. The reaction mixture was cooled to 35° C. and filtered. The product thus obtained was washed with diisopropyl ether and dried at 55° C. to 60° C. in hot air oven and cooled to 30° C. to 35° C. to obtain crystalline form of lomitapide free base.

### Example-9

### Preparation of Crystalline Form-I of Lomitapide Free Base

In a 500 mL round bottom flask, equipped with a mechanical stirrer, thermometer and an addition funnel, 10 g lomitapide base was dissolved in 20 mL ethylacetate at 35° C. The solution was heated at 95° C. and 200 mL n-heptane was added. The reaction mixture was cooled to 35° C. and filtered. The product thus obtained was washed with n-heptane and dried at 55° C. to 60° C. in hot air oven and cooled to 35° C. to obtain crystalline Form-I of lomitapide free base.

### Example-10

### Preparation of Crystalline Form-I of Lomitapide Free Base

In a 500 mL round bottom flask, equipped with a mechanical stirrer, thermometer and an addition funnel, 10 g lomitapide base and 30 mL isopropylacetate were heated at 65° C. to 70° C. to obtain the clear solution. The reaction mixture was cooled to 0° C. to 5° C. and filtered. The product thus obtained was washed with isopropylacetate and dried at 55° C. to 60° C. in hot air oven and cooled to 30° C. to 35° C. to obtain crystalline Form-I of lomitapide free base.

### Example-11

### Preparation of Crystalline Form-II of Lomitapide Free Base

In a 500 mL round bottom flask, equipped with a mechanical stirrer, thermometer and an addition funnel, 10 g lomitapide base and 30 mL toluene were heated at 105° C. to 110° C. to obtain the clear solution. The reaction mixture was cooled to 30° C. to 35° C. and filtered. The product thus obtained was washed with toluene and dried at 45° C. to 50° C. in vacuum tray dryer and cooled to 30° C. to 35° C. to obtain crystalline Form-II of lomitapide free base.

### Example-12

### Preparation of Crystalline Form-A of Lomitapide Mesylate

In a 500 mL round bottom flask, equipped with a mechanical stirrer, thermometer and an addition funnel, 10 g lomitapide mesylate and 50 mL isopropylacetate were heated at 65° C. to 70° C. to obtain the clear solution. The reaction mixture was cooled to 0° C. to 5° C. and filtered. The product thus obtained was washed with isopropylacetate and dried at 55° C. to 60° C. in hot air oven and cooled to 30° C. to 35° C. to obtain crystalline form of lomitapide mesylate.

### Example-13

### Preparation of Crystalline Form-B of Lomitapide Mesylate

In a 500 mL round bottom flask, equipped with a mechanical stirrer, thermometer and an addition funnel, 10 g lomitapide mesylate and 30 mL acetonitrile were heated at 75° C. to 80° C. to obtain the clear solution. The reaction mixture was cooled to 0° C. to 5° C. and filtered. The product thus obtained was washed with acetonitrile and dried at 55° C. to 60° C. in hot air oven and cooled to 30° C. to 35° C. to obtain crystalline Form-B of lomitapide mesylate.

### Example-14

### Preparation of Crystalline Form-C of Lomitapide Mesylate

10 g lomitapide mesylate and 30 mL isopropanol were heated in a 100 mL round bottom flask, equipped with a mechanical stirrer, thermometer and an addition funnel at 70-80° C. and stirred to obtain clear solution. The solution was cooled to 20-30° C. and the solid obtained was filtered, washed with isopropanol and dried to obtain the Form-C.

### Example-15

### Preparation of Crystalline Form-C of Lomitapide Mesylate

10 g lomitapide mesylate, 30 mL methylene dichloride and 20 mL n-heptane were heated in a 100 mL round bottom flask, equipped with a mechanical stirrer, thermometer and stirred to obtain clear solution. The solution was cooled to 15-20° C. and the solid obtained was filtered, washed with methylene dichloride and dried to obtain the Form-C.

While the present invention has been described in terms of its specific embodiments, certain modification and equivalents will be apartment to those skilled in the art and are intended to be included within the scope of the present invention.

1. Lomitapide mesylate in solid amorphous form.

2. The amorphous form according to claim 1 having less than about 0.5% residual solvents when measured by GC and having a moisture content less than 5% wt/wt.

3. The amorphous form according to claim 1 having a purity of about 98% or more, as measured by area percentage of HPLC.

4. The amorphous form according to claim 1 which is stable for a period of at least three months alter exposure to a relative humidity of 75% at 40° C. or a relative humidity of 60% at 25° C., and does not change to any crystalline form and contains less than about 0.5% wt/wt total impurities.

5. The lomitapide mesylate according to claim 1 having particle size distribution as characterized by 90% particles having particle size (D₉₀) of about 250 μm or less, 50% particles having particle size (D₅₀) of about 100 μm or less, and 10% particles having particle size (D₁₀) of about 50 μm or less.

6. A process for the preparation of the amorphous form of lomitapide mesylate according to claim 1, the process comprising:
(a) providing a solution of lomitapide mesylate in one or more solvents; and
(b) obtaining the amorphous form of lomitapide mesylate by the removal of the solvents, or
(a) providing a solution of lomitapide free base in one or more solvents;
(b) adding methane sulfonic acid to the solution; and
(c) obtaining the amorphous form of lomitapide mesylate by the removal of the solvents or by adding one or more of anti-solvents to the solution.

7. The process according to claim 6, wherein the solvent comprises one or more of alcohols selected from methanol, ethanol, n-propanol, isopropanol (IPA), and n-butanol; esters selected from ethyl acetate, propyl acetate, isopropyl acetate, t-butyl acetate, and isobutyl acetate; ketones selected from acetone, methyl ethyl ketone, and methyl isobutyl ketone; halogenated hydrocarbons selected from methylene dichloride, ethylene dichloride, carbon tetrachloride and chlorobenzene; polar aprotic solvent selected from dimethylformamide, dimethylsulfoxide, and N-methylpyrrolidone; tetrahydrofuran, 2-methyl tetrahydrofuran, dioxane, acetonitrile, or mixtures thereof.

8. The process according to claim 6, wherein the solvent is removed by one or more techniques selected from distillation, distillation under vacuum, evaporation, evaporation by rotational distillation, spray drying, agitated thin film drying (“ATFD”), freeze drying (lyophilization), filtration, decantation, and centrifugation.

9. The process according to claim 6, wherein the anti-solvent comprises one or more of diethylether, diisopropyether, methyl tert-butyl ether, tetrahydrofuran, 1,4-dioxane, hexane, heptane, octane, cyclohexane, toluene, xylene and ethylbenzene.

10. An amorphous solid dispersion of lomitapide mesylate and at least one pharmaceutically acceptable carrier.

11. The solid dispersion according to claim 10, wherein the pharmaceutically acceptable carrier is a polymer selected from a non-ionic or an ionic polymer comprising one or more of hydroxypropylmethyl cellulose acetate succinate (HPMC-AS), hydroxypropylmethyl cellulose (HPMC), methacrylic acid copolymers and polyvinylpyrrolidone (PVP).

12. A crystalline Form-A of lomitapide mesylate characterized by x-ray powder diffraction pattern having characteristic peaks expressed in terms of 2θ at about 6.3°, 12.6°, 14.1°, 16.2°, 21.7° and 23.7°±0.2° (2θ).

13. A crystalline Form-B of lomitapide mesylate characterized by x-ray powder diffraction pattern having characteristic peaks expressed in terms of 2θ at about 7.0°, 11.3°, 12.1°, 13.3°, 16.9°, 22.4° and 28.6°±0.2° (2θ).

14. A crystalline Form-C of lomitapide mesylate characterized by x-ray powder diffraction pattern having characteristic peaks expressed in terms of 2θ at about 3.9, 9.9, 15.5, 19.5 and 22.0°±0.2° (2θ).

15. A process for the preparation of crystalline form of lomitapide mesylate, the process comprising:
(a) providing a solution of lomitapide mesylate in one or more solvents; and
(b) obtaining the crystalline form of lomitapide mesylate by the removal of the solvents.

16. The process according to claim 15, wherein the crystalline form of lomitapide mesylate having a purity of about 98% or more, as measured by area percentage of HPLC.

17. The process according to claim 15, wherein the crystalline form of lomitapide mesylate having particle size distribution as characterized by 90% particles having particle size (D₉₀) of about 250 μm or less, 50% particles having particle size (D₅₀) of about 100 μm or less, and 10% particles having particle size (D₁₀) of about 50 μm or less.

18. The process according to claim 15, wherein the solvent comprises one or more of alcohols selected from methanol, ethanol, n-propanol, isopropanol (IPA), and n-butanol; esters selected from ethyl acetate, propyl acetate, isopropyl acetate, t-butyl acetate, and isobutyl acetate; ketones selected from acetone, methyl ethyl ketone, and methyl isobutyl ketone; halogenated hydrocarbons selected from methylene dichloride, ethylene dichloride, carbon tetrachloride and chlorobenzene; polar aprotic solvents selected from dimethylformamide, dimethylsulfoxide, and N-methylpyrrolidone; acetonitrile, or mixture thereof.

19. The process according to claim 15, wherein the solvent is removed by one or more techniques selected from filtration, decantation, and/or centrifugation.

20. The process according to claim 15, wherein the crystalline form of lomitapide mesylate is the Form-A of lomitapide mesylate characterized by x-ray powder diffraction pattern having characteristic peaks expressed in terms of 2θ at about 6.3°, 12.6°, 14.1°, 16.2°, 21.7° and 23.7°±0.2° (2θ) which further comprises:
(a) providing a solution of lomitapide mesylate in one or more ester solvent;
(b) heating the solution; and
(c) obtaining the crystalline Form-A of lomitapide mesylate by the removal of the solvent.

21. The process according to claim 20, wherein the ester solvent comprises one or more of ethyl acetate, propyl acetate, isopropyl acetate, butyl acetate and isobutyl acetate.

22. The process according to claim 15, wherein the crystalline form of lomitapide mesylate is the Form-B of lomitapide mesylate characterized by x-ray powder diffraction pattern having characteristic peaks expressed in terms of 2θ at about 7.0°, 11.3°, 12.1°, 13.3°, 16.9°, 22.4° and 28.6°±0.2° (2θ) which further comprises:
(a) providing a solution of lomitapide mesylate in a nitrile solvent;
(b) heating the solution; and
(c) obtaining the crystalline Form-B of lomitapide mesylate by the removal of the solvent.

23. The process according to claim 22, wherein the nitrile solvent comprises one or more of acetonitrile and propionitrile.

24. The process according to claim 15, wherein the crystalline firm of lomitapide mesylate is Form-C of lomitapide mesylate characterized by x-ray powder diffraction pattern having characteristic peaks expressed in terms of 2θ at about 3.9, 9.9, 15.5, 19.5 and 22.0°±0.2° (2θ) which further comprises:
(a) providing a solution of lomitapide mesylate in a solvent selected from alcoholic solvents, halogenated solvents, hydrocarbons or mixtures thereof; and
(b) obtaining the crystalline Form-C of lomitapide mesylate by the removal of the solvents.

25. The process according to claim 24, wherein the alcoholic solvent is selected from methanol, ethanol, n-propanol, isopropanol (IPA), and n-butanol; halogenated solvent is selected from methylene dichloride, carbon tetrahydrochloride and chloroform; hydrocarbons is selected from n-hexane, n-heptane, cyclohexane, toluene and xylene.

26. Lomitapide free base in solid amorphous form.

27. A process for the preparation of the amorphous form of lomitapide free base according to claim 26, the process comprising:
(a) providing a solution of lomitapide free base in one or more solvents; and
(b) obtaining the amorphous form of lomitapide free base by the removal of the solvents, or by adding one or more of anti-solvents to the solution.

28. The process according to claim 27, wherein the solvent comprises one or more of alcohols selected from methanol, ethanol, n-propanol, isopropanol (IPA), and n-butanol; esters selected from ethyl acetate, propyl acetate, isopropyl acetate, t-butyl acetate, and isobutyl acetate; ketones selected from acetone, methyl ethyl ketone, and methyl isobutyl ketone; ethers selected from diethylether, diisopropyl ether, methyl tert-butyl ether, tetrahydrofuran, 2-methyltetrahydrofura, 1,4-doxane, halogenated hydrocarbons selected from methylene dichloride, ethylene dichloride, carbon tetrachloride and chlorobenzene; aromatic hydrocarbons selected from toluene, xylene, chlorobenzene and ethylbenzene, polar aprotic solvents selected from dimethylformamide, dimethylsulfoxide, and N-methylpyrrolidone, or mixture thereof.

29. The process according to claim 27, wherein the solvent is removed by one or more techniques selected from distillation, distillation under vacuum, evaporation, evaporation by rotational distillation, spray drying, agitated thin film drying (“ATFD”), freeze drying (lyophilization), filtration, decantation, and centrifugation.

30. The process according to claim 27, wherein the anti-solvent comprises one or more of water, hexane, heptane, octane and cyclohexane.

31. A crystalline Form-I of lomitapide free base characterized by x-ray powder diffraction pattern having characteristic peaks expressed in terms of 2θ at about 5.4°, 10.8°, 13.6°, 21.8°, and 31.3°±0.2° (2θ).

32. A process for the preparation of a crystalline Form-I of lomitapide free base according to claim 31, the process comprising:
(a) providing a solution of lomitapide free base in one or more solvents; and
(b) obtaining the crystalline Form-I of lomitapide free base by the removal of the solvent.

33. The process according to claim 32, wherein the solvent comprises one or more of alcohols selected from methanol, ethanol, n-propanol, isopropanol (IPA), and n-butanol; esters selected from ethyl acetate, propyl acetate, isopropyl acetate, t-butyl acetate, and isobutyl acetate; ketones selected from acetone, methyl ethyl ketone, and methyl isobutyl ketone; ethers selected from diethyl ether, diisopropyl ether, methyl tert-butyl ether, tetrahydrofuran, 2-methyltetrahydrofuran, and 1,4-doxane; halogenated hydrocarbons selected from methylene dichloride, ethylene dichloride, carbon tetrachloride and chlorobenzene; polar aprotic solvent selected from dimethylformamide, dimethylsulfoxide, and N-methylpyrrolidone or mixtures thereof.

34. A process for the preparation of crystalline Form-I of lomitapide free base according to claim 31, the process comprising:
(a) providing a solution of lomitapide free base in one or more solvents;
(b) heating the solution; and
(c) obtaining the crystalline Form-I of lomitapide free base by adding one or more anti-solvents.

35. The process according to claim 34, wherein the anti-solvent is selected from one or more of water, hexane, heptane, octane, cyclohexane, toluene, xylene and ethylbenzene.

36. A crystalline Form-II of lomitapide free base characterized by x-ray powder diffraction pattern having characteristic peaks expressed in terms of 2θ at about 9.3°, 18.0°, 19.1°, 21.1°, and 23.9°±0.2° (2θ).

37. A process for the preparation of a crystalline Form-II of lomitapide free base according to claim 36, the process comprising:
(a) providing a solution of lomitapide free base in one or more hydrocarbon solvents; and
(b) obtaining the crystalline Form-II of lomitapide free base by the removal of the solvent.

38. The process according to claim 37, wherein the hydrocarbon solvent is selected from one or more of toluene, xylene or ethylbenzene.

39. A pharmaceutical composition comprising the amorphous form of lomitapide mesylate according to claim 1 and pharmaceutically acceptable carriers, diluents and excipients.

40. A pharmaceutical composition comprising the crystalline form of lomitapide mesylate according to claim 12 and pharmaceutically acceptable carriers, diluents and excipients.

41. A pharmaceutical composition comprising the crystalline form of lomitapide mesylate according to claim 13 and pharmaceutically acceptable carriers, diluents and excipients.

42. A pharmaceutical composition comprising the crystalline form of lomitapide mesylate according to claim 14 and pharmaceutically acceptable carriers, diluents and excipients.

43. A pharmaceutical composition comprising the amorphous form of lomitapide free base according to claim 26 and pharmaceutically acceptable carriers, diluents and excipients.

44. A pharmaceutical composition comprising the crystalline form of lomitapide free base according to claim 31 and pharmaceutically acceptable carriers, diluents and excipients.

45. A pharmaceutical composition comprising the crystalline form of lomitapide free base according to claim 36 and pharmaceutically acceptable carriers, diluents and excipients.
